(19) United States
(12) Patent Application Publication
(10) Pub. No.: US 2008/0255103 A1
(43) Pub. Date: Oct. 16, 2008

(54) Antihistamine and anti-nausea pharmaceutical compositions for topical application

(75) Inventors: Mohammad Aslam, Porter, TX (US); J. Rao Nulu, Austin, TX (US); Terry Ballay, San Marcos, TX (US); Matthew S. Ballay, Kyle, TX (US)

Correspondence Address: AKIN, GUMP, STRAUSS, HAUER & FELD, L.L.P., P.O. BOX 12870, SAN ANTONIO, TX, 78212, US

(73) Assignee: Ballay Pharmaceuticals, Inc., Wimberley, TX (US)

(21) Appl. No.: 11/786,715

(22) Filed: Apr. 12, 2007

(51) Int. Cl. A61K 31/54; A61P 1/08; A61P 37/00

(52) U.S. Cl. 514/224.8

(57) ABSTRACT

A topical pharmaceutical preparation suitable for use as an antihistamine or anti-nausea drug comprises an emu oil gel base and a therapeutic amount of promethazine.HCl. The preparation may further comprise an antioxidant. The preparation is preferably packaged as a single unit dose. A method for preparing the topical pharmaceutical preparation comprises the steps of combining a clear gel with an emu oil mixture and adding a therapeutic amount of promethazine.HCl solution.

### FIELD OF THE INVENTION

This invention relates to pharmaceuticals, and more particularly to a topical pharmaceutical preparation suitable for use as an antihistamine or anti-nausea drug.

### BACKGROUND OF THE INVENTION

It is desirable to create a preparation of an antihistamine and anti-nausea drug for topical application that is shelf stable, readily absorbed through a patient's skin, and packaged as a single unit dose. In the pharmaceutical industry, prescription drugs are often available in various forms, including pills and liquids for oral administration, injections, suppositories, and topically applied creams and gels. One such drug is promethazine or its commonly prescribed salt, promethazine hydrochloride (promethazine.HCl), an antihistamine and anti-nausea drug.

Currently, promethazine.HCl is commercially available as a pill, suppository, syrup, and injectable. Some patients are unable to or prefer not to swallow the drug, use suppositories, or inject the drug. A topically applied preparation of promethazine.HCl would allow for absorption of the drug through the patient's skin, also known as transdermal delivery, thereby avoiding these potentially undesirable administration methods.

However, when promethazine.HCl is compounded with standard cream or gel bases such as petroleum jelly or water at compounding pharmacies, the preparation is unstable and quickly oxidizes, turning a dark blue or other unpleasant, dark color due to thermal or photochemical decomposition over time. This unappealing characteristic makes the cream or gel commercially unacceptable, particularly because it may prevent patients from using the drug as prescribed. For instance, a patient who purchases a white promethazine.HCl cream or gel from the pharmacy, which subsequently turns a dark color, is likely to stop using the product, justifiably believing that something was wrong with the medication. Even if the promethazine.HCl preparation was a dark color at the time of purchase, the patient is unlikely to apply a dark blue product to his or her skin, as opposed to a white cream. This problem of patient intolerance to current promethazine.HCl creams and gels poses a health risk by encouraging patient non-compliance with physician directives.

Moreover, the risk of misapplication and overdose is particularly high with topically applied drugs if they are not provided in well designed unit doses. It is difficult for a patient to accurately measure the amount of cream or gel dispensed from large tubes to apply exactly prescribed doses to the skin, thereby increasing the chance for over- or under-dosing. And when localized application of the drug is prescribed, it is difficult for the patient to know how much of the drug is absorbed through the skin on the hand used to apply the drug, rather than the target area.

The components of a topically applied drug can also affect the dosage actually delivered through the patient's skin. Certain carrier components in the preparation can increase transdermal delivery of the drug, as can a greater surface area of application. On the other hand, more localized application of a preparation without any carrier components can decrease the amount of the drug delivered into the bloodstream.

Previous attempts at achieving reliable transdermal delivery of promethazine have not adequately addressed these problems of shelf-stability and proper dosage. For example, published U.S. Pat. App. No. 2004/258,740 to Thompson teaches a solid tablet with an effervescent agent and an acid, such as citric acid, that allows an active ingredient in the tablet to rapidly disperse in water. The affected area is then soaked in the treated water, thereby increasing surface area contact between the patient's skin and the active ingredient, with the objective of increasing transdermal delivery of the active ingredient. But the effervescent tablet preparation of Thompson requires access to water and time to soak the affected area. It does not address the need for a commercially attractive, shelf-stable cream or gel preparation of promethazine.HCl.

Similarly, published U.S. Pat. App. No. 2005/69,566 to Tamarkin et al. discloses a foam formulation using a liquefied gas propellant for transdermal delivery of various active ingredients, including promethazine.HCl. But the Tamarkin et al. foam does not address the concern of proper dosage with topically applied pharmaceuticals, and more specifically, the foam carrier of Tamarkin et al. does not teach a shelf-stable cream or gel preparation of promethazine.HCl for topical application.

What is needed in the pharmaceutical industry is a commercially attractive, patient-tolerated, and shelf-stable preparation of an antihistamine and anti-nausea drug for topical application that is packaged as a single unit dose.

### SUMMARY OF THE INVENTION

In accordance with one aspect of the present invention, a topical pharmaceutical preparation suitable for use as an antihistamine or anti-nausea drug comprises an emu oil gel base and a therapeutic amount of promethazine.HCl. The presence of emu oil in the present preparation optimizes absorption of promethazine.HCl through the skin without the necessity of applying the formula over a large area of the skin. The emu oil gel base also maintains the promethazine.HCl preparation at a commercially acceptable and patient-tolerated, consistent white color not found in prior-art preparations.

In accordance with a further aspect of the present invention, a topical pharmaceutical preparation suitable for use as an antihistamine or anti-nausea drug comprises an emu oil gel base, a therapeutic amount of promethazine.HCl, and an antioxidant. The antioxidant may preferably be alpha-lipoic acid. The presence of alpha-lipoic acid may provide further beneficial stabilization of the promethazine.HCl preparation, maintaining the white color not found in prior-art formulations for topical promethazine.HCl delivery, even upon exposure to heat and sunlight.

In accordance with an even further aspect of the invention, the topical pharmaceutical preparation of the present invention is packaged as a single unit dose. The preparation may be packaged in small ampoules or other collapsible containers of suitable materials, with each ampoule or container holding a single, prescribed dose of the topical preparation.

In accordance with a still further aspect of the invention, a method of preparing a topical pharmaceutical preparation suitable for use as an antihistamine or anti-nausea drug comprises the steps of combining gel components with an emu oil mixture and adding a therapeutic amount of promethazine.HCl solution. The method of preparing a topical pharmaceutical preparation may also comprise the steps of adding aloe vera juice and triethanolamine. Most preferably, the method further comprises the step of packaging the topical pharmaceutical preparation as a single unit dose.

### BRIEF DESCRIPTION OF THE DRAWINGS

The foregoing summary, as well as the following detailed description of preferred embodiments of the invention, will be better understood when read in conjunction with the appended drawings. For the purpose of illustrating the invention, two embodiments of the single unit dose package that are presently preferred are shown in the drawings. It should be understood, however, that the invention is not limited to the precise arrangements and instrumentalities shown. In the drawings:

FIG. 1 is a side view of a preferred single unit dose package in accordance with one embodiment of the present invention.

FIG. 2 is a view of a second preferred single unit dose package in accordance with a second embodiment of the present invention.

### DETAILED DESCRIPTION OF THE PREFERRED EMBODIMENTS

A preparation of an antihistamine and anti-nausea drug for topical application according to a first embodiment of the present invention includes an emu oil gel base and promethazine.HCl. The emu oil gel base comprises emu oil to emulsify and aid in absorption of promethazine.HCl. More preferably, the emu oil gel base comprises emu oil, purified water, and potassium sorbate and methyl paraben sodium to prevent mold and bacteria growth, respectively. The emu oil gel base preferably further comprises sodium citrate as a stabilizer, disodium EDTA, Carbopol® Ultrez 10 (Noveon, Inc., Cleveland, Ohio), Tween® 80 (ICI Americas, Inc., Bridgewater, N.J.), propylene glycol to improve texture, glycerin as an emollient, and xantham gum to thicken the preparation. Preferably, triethanolamine is added to the emu oil gel base to thicken the preparation and to create a gel consistency. It is preferable for the promethazine.HCl to be dissolved in water with aloe vera juice. The aloe vera juice may preferably be prepared by adding aloe vera powder to water.

Further to this first embodiment, promethazine.HCl is present at a level preferably from about 0.1% to about 20%, although the lower bound is flexible to the extent that the resulting preparation contains a therapeutic amount of promethazine.HCl. More preferably, promethazine.HCl is present at a level from about 0.5% to about 15%, and most preferably from about 1.25% to about 10% by weight. The preparation according to this first embodiment preferably includes emu oil at a level from about 0.1% to about 20%, more preferably from about 1% to about 10%, and most preferably at about 5% by weight.

According to a second embodiment of the present invention, a preparation of an antihistamine and anti-nausea drug for topical application includes an emu oil gel base, promethazine.HCl, and an antioxidant. The emu oil gel base includes emu oil and components known in the art of pharmaceutical creams and gels for application to the skin. Preferably, the emu oil gel base comprises emu oil, purified water, potassium sorbate, methyl paraben sodium, sodium citrate, disodium EDTA, Carbopol® Ultrez 10, Tween® 80, xantham gum, propylene glycol, and glycerin. The emu oil gel base preferably also includes triethanolamine to thicken the preparation and to make a gel consistency. It is preferable for the promethazine.HCl to be dissolved in purified water and aloe vera juice. The aloe vera juice may preferably be prepared by adding aloe vera powder to water. The antioxidant according to this second embodiment of the present invention may be selected from a number of suitable antioxidants known in the art, but preferably comprises alpha-lipoic acid. Less preferred antioxidants comprise ascorbyl palmitate, ascorbic acid, vitamin E, and hydroquinone.

Further to this second embodiment of the present invention, promethazine.HCl is present at a level preferably from about 0.1% to about 20%, more preferably from about 0.5% to about 15%, and most preferably from about 1.25% to about 10% by weight. Emu oil is present at a level preferably from about 0.1% to about 20%, more preferably from about 1% to about 10%, and most preferably at about 5% by weight. And alpha-lipoic acid is present at a level preferably from about 0.01% to about 5%, and most preferably from about 0.01% to about 1% by weight.

Further still to both the first and second embodiments of the present invention, the inventive preparations for topical application maintain a shelf-stable, patient-tolerated, and commercially acceptable white color, even after more than ninety days of storage at ambient temperatures. The same white color is maintained after prolonged storage at 35° Celsius.

According to a third embodiment of the present invention, a preparation of an antihistamine and anti-nausea drug for topical application is prepared by combining a clear gel with an emu oil mixture and adding a promethazine.HCl solution. Preferably, the clear gel is prepared by mixing purified water, potassium sorbate, methyl paraben sodium, sodium citrate, disodium EDTA, Carbopol® Ultrez 10, and xantham gum. The emu oil mixture preferably comprises emu oil and Tween® 80, and in other preferred embodiments further comprises propylene glycol, glycerin, alpha-lipoic acid, or any combination of these components. The promethazine.HCl solution preferably comprises promethazine.HCl in purified water and aloe vera juice. Most preferably, the preparation of the present invention is further prepared by adding triethanolamine to create a thick gel consistency.

In accordance with a fourth embodiment of the present invention, the preparation of the present invention is packaged as a single unit dose. Preferably, the single unit dose packaging comprises small tubes or other collapsible containers manufactured of suitable materials, each tube or other container holding a prescribed dose of the topical preparation. More preferably, the single unit dose packaging comprises a plastic or foil material, filled with between about 0.10 mL and about 1.0 mL of the topical promethazine.HCl preparation, and even more preferably, between about 0.25 mL and 0.50 mL of the topical promethazine.HCl preparation. These preferred embodiments preferably provide a unit dose of about 0.625 mg to about 50 mg of promethazine.HCl, and more preferably of about 25 mg to about 50 mg of promethazine.HCl.

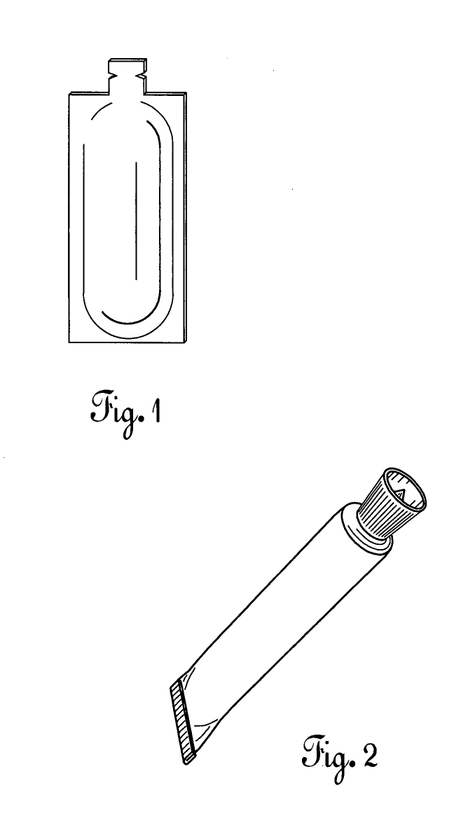

Referring now to FIGS. 1 and 2, the topical promethazine.HCl preparation may preferably be packaged using a blow-fill-seal process in which the unit dose package is formed, the topical promethazine.HCl preparation is injected into the package, and the unit dose package is sealed. The topical promethazine.HCl preparation may also preferably be packaged in a foil container in which the foil is welded to form a pouch as the topical promethazine.HCl preparation is introduced into the pouch. The unit dose packaging preferably has a twist-off opening capability that liberates the topical promethazine.HCl preparation upon twisting. FIG. 1. It is equally preferable for the unit dose packaging to be closed by a screw cap that may be used to pierce the unit dose packaging for liberating the topical promethazine.HCl preparation. FIG. 2.

The single unit dose packaging most preferably ensures that patients accurately dispense and apply one full dose of the present preparation to minimize misapplication and overdose This objective may further be achieved by color-coding the outer material of the unit dose packaging to identify the promethazine.HCl concentration of the contained topical promethazine.HCl preparation.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

### Example 1

### Promethazine.HCl Gel (0.625%) with Alpha-Lipoic Acid (0.1%)

Approximately 100 mL purified water is mixed with 0.2 g potassium sorbate, 0.4 g methyl paraben sodium, 1.0 g sodium citrate, and 1.0 g disodium EDTA. To this clear solution, 3.0 g Carbopol® Ultrez 10 and 0.6 g xantham gum are added and the mixture is stirred with a mechanical stirrer. In a separate glass container, 0.2 g alpha-lipoic acid, 0.6 g Tween®-80, 6.0 g propylene glycol, and 10.0 g emu oil are mixed and heated to 40° C. This mixture is added to the above gel solution. 1.25 g promethazine.HCl is mixed well in 50 mL purified water and added to the above solution with 6 mL aloe vera juice. The pH of the resulting mixture is 3.5. 8.0 g triethanolamine is added to thicken the gel. The resulting pH of the gel is 6.5. The gel is stored in polypropylene and amber glass jars.

When prepared, the gel is white, and the gel remains white at ambient temperatures.

### Example 2

### Promethazine.HCl Gel (1.25%) with Alpha-Lipoic Acid (1.0%)

Approximately 100 mL of purified water is mixed with 0.2 g potassium sorbate, 0.4 g methyl paraben sodium, 1.0 g sodium citrate, and 1.0 g disodium EDTA. To this clear mixture is added 3.0 g Carbopol® Ultrez 10 and 0.6 g xantham gum and the mixture is stirred with a mechanical mixer. In a suitable glass container 2.0 g alpha-lipoic acid, 0.6 g Tween® 80, 10.0 g emu oil, 8.0 g propylene glycol, and 8.0 g glycerin are mixed and heated to 40° C. This hot mixture is added to the above gel solution. Then 2.5 g promethazine.HCl, 6.0 mL aloe vera juice, and 10 mL purified water are added to the above gel, and the pH of this gel mixture is 3.5. 4.0 g triethanolamine is added to make a thick gel. Sufficient purified water is added to make 200 g and stirred very well to make a gel. The resulting pH of the gel is 6.5. The gel is stored in white polypropylene and amber jars.

The gel when prepared is white, and the gel remains white at ambient temperatures.

### Example 3

### Promethazine.HCl Gel (2.5%) with Alpha-Lipoic Acid (0.2%)

Approximately 100 mL purified water is mixed with 0.2 g potassium sorbate, 0.4 g methyl paraben sodium, 1.0 g sodium citrate, and 1.0 g disodium EDTA. To this clear mixture is added 8.0 g Carbopol® Ultrez 10 and 0.6 g xantham gum and stirred with a mechanical mixer. In a suitable glass container 0.4 g alpha-lipoic acid, 0.6 g Tween®-80, 8.0 g propylene glycol, 8.0 g glycerin, and 10.0 g emu oil are mixed and heated to 40° C. This hot mixture is added to the above gel solution. Then 5.0 g promethazine.HCl in 20 mL purified water is added and mixed well. 6.0 mL of aloe vera juice is added, and the pH of the resulting gel mixture is 3.5. 8.0 g triethanolamine is added to thicken the gel. Sufficient purified water is added to make 200 g and stirred very well to make a gel. The resulting pH of the gel is 6.5. The gel is stored in white polypropylene and amber jars.

The gel when prepared is white, and the gel remains white at ambient temperatures.

### Example 4

### Promethazine.HCl Gel (5.0%)

At about 40° C, approximately 300 mL purified water is mixed with 0.5 g potassium sorbate, 1.0 g methyl paraben sodium, 2.5 g sodium citrate, and 2.5 g disodium EDTA. To this clear mixture is added 15.0 g Carbopol® Ultrez 10 and 1.5 g xantham gum and stirred with a mechanical mixer. To the above gel mixture, 25.0 g promethazine.HCl and 15.0 mL aloe vera juice are added. In a suitable glass container, 1.0 g Tween® 80, 20.0 g propylene glycol, 25.0 g emu oil, and 20.0 g glycerin are mixed and heated to 45° C. This hot mixture is added to the above gel solution, and the pH of the resulting gel mixture is 3.5. 20.0 g triethanolamine is added to thicken the gel. Sufficient purified water is added to make 500 g and stirred very well to make a thick gel. The resulting pH of the gel is 6.6. The gel is stored in white polypropylene and amber jars.

The gel when prepared is white, and the gel remains white at ambient temperatures.

### Example 5

### Promethazine.HCl Gel (5.0%) with Alpha-Lipoic Acid (0.1%)

At about 40° C., approximately 300 mL purified water is mixed with 0.5 g potassium sorbate, 1.0 g methyl paraben sodium, 2.5 g sodium citrate, and 2.5 g disodium EDTA. To this clear mixture is added 15.0 g Carbopol® Ultrez 10 and 1.5 g xantham gum and stirred with a mechanical mixer. To the above gel mixture, 25.0 g promethazine.HCl and 15.0 mL aloe vera juice are added. In a suitable glass container, 0.5 g alpha-lipoic acid, 1.0 g Tween® 80, 20.0 g propylene glycol, 25.0 g emu oil, and 20.0 g glycerin are mixed and heated to 45° C. This hot mixture is added to the above gel solution, and the pH of the resulting gel mixture is 3.5. 20.0 g triethanolamine is added to thicken the gel. Sufficient purified water is added to make 500 g and stirred very well to make a thick gel. The resulting pH of the gel is 6.6. The gel is stored in white polypropylene and amber jars.

The gel when prepared is white, and the gel remains white at ambient temperatures.

### Example 6

### Promethazine.HCl Gel (10.0%)

At about 40° C., approximately 300 mL purified water is mixed with 0.5 g potassium sorbate, 1.0 g methyl paraben sodium, 2.5 g sodium citrate, and 2.5 g disodium EDTA. To this clear mixture is added 15.0 g Carbopol® Ultrez 10 and 1.5 g xantham gum and stirred with a mechanical mixer. To the above gel mixture, 50.0 g promethazine.HCl and 15.0 mL aloe vera juice are added. In a suitable glass container, 1.0 g Tween® 80, 20.0 g propylene glycol, 25.0 g emu oil, and 20.0 g glycerin are mixed and heated to 45° C. This hot mixture is added to the above gel solution, and the pH of the resulting gel mixture is 3.5. 35.0 g triethanolamine is added to thicken the gel. Sufficient purified water is added to make 500 g and stirred very well to make a thick gel. The resulting pH of the gel is 6.6. The gel is stored in white polypropylene and amber jars.

The gel when prepared is white, and the gel remains white at ambient temperatures.

### Example 7

### Promethazine.HCl Gel (10.0%) with Alpha-Lipoic Acid (0.1%)

At about 40° C., approximately 300 mL purified water is mixed with 0.5 g potassium sorbate, 1.0 g methyl paraben sodium, 2.5 g sodium citrate, and 2.5 g disodium EDTA. To this clear mixture is added 15.0 g Carbopol® Ultrez 10 and 1.5 g xantham gum and stirred with a mechanical mixer. To the above gel mixture, 50.0 g promethazine.HCl and 15.0 mL aloe vera juice are added. In a suitable glass container, 0.5 g alpha-lipoic acid, 1.0 g Tween® 80, 20.0 g propylene glycol, 25.0 g emu oil, and 20.0 g glycerin are mixed and heated to 45° C. This hot mixture is added to the above gel solution, and the pH of the resulting gel mixture is 3.5. 35.0 g triethanolamine is added to thicken the gel. Sufficient purified water is added to make 500 g and stirred very well to make a thick gel. The resulting pH of the gel is 6.6. The gel is stored in white polypropylene and amber jars.

The gel when prepared is white, and the gel remains white at ambient temperatures.

It will be appreciated by those skilled in the art that changes could be made to the embodiments described above without departing from the broad inventive concept thereof. By way of example, the emu oil gel base may be combined with other pharmacologically active components to create shelf-stable preparations for topical application, or emu oil may be combined with other well-known cream or gel components to create shelf-stable topical formulas of different consistencies. It will be understood, therefore, that this invention is not limited to the particular embodiments disclosed, but it is intended to cover modifications within the spirit and scope of the present invention as defined by the appended claims.

What is claimed is:

1. A topical pharmaceutical preparation suitable for use as an antihistamine or anti-nausea drug comprising an emu oil gel base and a therapeutic amount of promethazine.HCl.

2. The preparation of claim 1 wherein said emu oil gel base comprises emu oil and a gelling or neutralizing agent.

3. The preparation of claim 2 wherein said gelling or neutralizing agent comprises triethanolamine.

4. The preparation of claim 1 wherein said promethazine.HCl comprises from about 0.1% to about 20% by weight of the preparation.

5. The preparation of claim 1 wherein said promethazine.HCl comprises from about 0.5% to about 15% by weight of the preparation.

6. The preparation of claim 1 wherein said promethazine.HCl comprises from about 1.25% to about 10% by weight of the preparation.

7. The preparation of claim 2 wherein said emu oil comprises from about 0.1% to about 20% by weight of the preparation.

8. The preparation of claim 2 wherein said emu oil comprises from about 1% to about 10% by weight of the preparation.

9. The preparation of claim 2 wherein said emu oil comprises about 5% by weight of the preparation.

10. A topical pharmaceutical preparation for use as an antihistamine or anti-nausea drug and for encouraging patient compliance and preventing patient overdose comprising a single unit dose of a therapeutic amount of promethazine.HCl in an emu oil gel base.

11. The topical pharmaceutical preparation of claim 10 wherein said therapeutic amount comprises from about 0.625 mg to about 50 mg promethazine.HCl.

12. A topical pharmaceutical preparation suitable for use as an antihistamine or anti-nausea drug comprising an emu oil gel base, a therapeutic amount of promethazine.HCl, and an antioxidant.

13. The preparation of claim 12 wherein said emu oil gel base comprises emu oil.

14. The preparation of claim 12 wherein said emu oil gel base comprises emu oil and a gelling or neutralizing agent.

15. The preparation of claim 14 wherein said gelling or neutralizing agent comprises triethanolamine.

16. The preparation of claim 12 wherein said preparation is provided as a single unit dose.

17. The preparation of claim 12 wherein said promethazine.HCl comprises from about 0.1% to about 20% by weight of the preparation.

18. The preparation of claim 12 wherein said promethazine.HCl comprises from about 0.5% to about 15% by weight of the preparation.

19. The preparation of claim 12 wherein said promethazine.HCl comprises from about 1.25% to about 10% by weight of the preparation.

20. The preparation of claim 13 wherein said emu oil comprises from about 0.1% to about 20% by weight of the preparation.

21. The preparation of claim 13 wherein said emu oil comprises from about 1% to about 10% by weight of the preparation.

22. The preparation of claim 13 wherein said emu oil comprises about 5% by weight of the preparation.

23. The preparation of claim 14 wherein said emu oil comprises from about 0.1% to about 20% by weight of the preparation.

24. The preparation of claim 14 wherein said emu oil comprises from about 1% to about 10% by weight of the preparation.

25. The preparation of claim 14 wherein said emu oil comprises about 5% by weight of the preparation.

26. A topical pharmaceutical preparation suitable for use as an antihistamine or anti-nausea drug comprising an emu oil gel base, a therapeutic amount of promethazine.HCl, and alpha-lipoic acid.

27. The preparation of claim 26 wherein said alpha-lipoic acid comprises from about 0.1% to about 5% by weight of the preparation.

28. The preparation of claim 26 wherein said alpha-lipoic acid comprises from about 0.1% to about 1% by weight of the preparation.

29. A topical pharmaceutical preparation for use as an antihistamine or anti-nausea drug and for encouraging patient compliance and preventing patient overdose comprising a single unit dose of a therapeutic amount of promethazine.HCl in an emu oil gel base with alpha-lipoic acid.

30. The topical pharmaceutical preparation of claim 29 wherein said therapeutic amount comprises from about 0.625 mg to about 50 mg promethazine.HCl.

31. A method of preparing a topical pharmaceutical preparation suitable for use as an antihistamine or anti-nausea drug comprising the steps of (i) combining gel components with an emu oil mixture; and (ii) adding a therapeutic amount of promethazine.HCl solution.

32. A method of preparing a topical pharmaceutical preparation suitable for use as an antihistamine or anti-nausea drug comprising the steps of (i) combining gel components with an emu oil mixture; (ii) adding a therapeutic amount of promethazine.HCl solution; (iii) thickening the gel with a gelling or neutralizing agent; and (iv) providing said preparation as a single unit dose.

33. The method of claim 32 wherein said providing step is accomplished through the use of a single unit dose package that contains from about 0.625 mg to about 50 mg promethazine.HCl.

34. The method of claim 31 wherein said gel components comprise purified water, potassium sorbate, methyl paraben sodium, sodium citrate, disodium EDTA, Carbopol® Ultrez 10, xantham gum, propylene glycol, and glycerin.

35. The method of claim 31 wherein said emu oil mixture comprises emu oil and Tween® 80.

36. The method of claim 35 wherein said emu oil mixture further comprises propylene glycol and glycerin.

37. The method of claim 35 wherein said emu oil mixture further comprises alpha-lipoic acid.

38. The method of claim 31 wherein said promethazine.HCl solution comprises promethazine.HCl and purified water.

39. The method of claim 36 wherein said promethazine.HCl solution comprises promethazine.HCl and purified water.

40. The method of claim 31 further comprising the step of adding aloe vera juice.

41. The method of claim 31 further comprising the step of adding triethanolamine.

42. The method of claim 32 further comprising the step of adding aloe vera juice.

43. The method of claim 32 further comprising the step of adding triethanolamine.
